(19) United States
(12) Patent Application Publication
(10) Pub. No.: US 2012/0158074 A1
(43) Pub. Date: Jun. 21, 2012

(54) Fitted garment comprising heart monitoring with defibrillation capability

(75) Inventor: John Edward Hall, Charlottesville, VA (US)

(73) Assignee: MEDICINE HALL OF AMERICA, INC., Charlottesville, VA (US)

(21) Appl. No.: 13/373,379

(22) Filed: Nov. 14, 2011

Related U.S. Application Data
(60) Provisional application No. 61/459,759, filed on Dec. 20, 2010.

(51) Int. Cl. A61N 1/39

(52) U.S. Cl. 607/5

(57) ABSTRACT

The fitted garment is an undergarment for containing heart monitoring and defibrillation devices that is interchangeable, washable and cleanable. The fitted garment is capable of being attached to a stylish and fashionable outer garment which carries and conceals the various connectors and electrodes.

### REFERENCES CITED

### U.S. PATENTS

| | | | |
|---|---|---|---|
| 1,092,144 | September 1914 | S. Grushlaw | — |
| 2,008,773 | July 1935 | C. H. Shapiro | 2/80 |
| 2,156,504 | May 1939 | B. S. Liss | 2/102 |
| 2,479,246 | August 1949 | B. Lupo | 2/114 |
| 2,688,752 | September 1954 | D. G. Sbarra | 2/113 |
| 2,704,070 | March 1955 | A. B. Rudisill | 128/167 |
| 2,918,677 | December 1959 | C. Pindyck | 2/111 |
| 3,409,007 | November 1968 | E. W. Fuller | 128/2.06 |
| 3,534,727 | October 1970 | J. A. Roman | 128/2.06 |
| 4,026,278 | March 1977 | Ricketts et al. | 128/2.06 |
| 4,087,864 | March 1978 | LaBove et al. | 2/102 |
| 4,411,267 | October 1983 | A. M. Heyman | 128/385 |
| 4,498,480 | February 1985 | J. L. Mortensen | 128/644 |
| 4,570,268 | February 1986 | J. J. Freeman | 2/114 |
| 4,580,572 | April 1986 | Granek et al. | 128/639 |
| 4,582,508 | April 1986 | W. F. Pavelka | 604/179 |
| 4,583,547 | April 1986 | Granek et al. | 128/639 |
| 4,608,987 | September 1986 | H. E. Mills | 128/639 |
| 4,666,432 | May 1987 | McNeish et al. | 604/174 |
| 4,698,848 | October 1987 | M. C. Buckley | 2/114 |
| 4,729,377 | March 1988 | Granek et al. | 128/639 |
| 4,889,131 | December 1989 | R. J. Salem et al | 128/671 |
| 5,007,427 | April 1991 | Suzuki et al. | 128/659 |
| 5,224,479 | July 1993 | Y. Sekine | 128/644 |
| 5,611,085 | March 1997 | V. Rasmussen | 2/102 |
| 5,708,978 | January 1998 | A. C. Johnsrud | 2/102 |
| 5,799,330 | September 1998 | C. O'Donoghue-Kitt | 2/114 |
| 5,806,096 | September 1998 | J. R. Pennington | 2/80 |
| 6,065,154 | May 2000 | Hukings et al. | 2/102 |
| 6,280,461 | August 2001 | Glegyak et al. | 607/5 |
| 6,289,238 | September 2001 | Besson et al. | 600/509 |
| 6,295,466 | September 2001 | Ishikawa et al. | 600/509 |
| 6,301,502 | October 2001 | Owen et al. | 607/5 |
| 6,304,780 | October 2001 | Owen et al. | 607/5 |
| 6,374,138 | April 2002 | Owen et al. | 607/5 |
| 6,388,422 | May 2002 | A. L. Lew | 320/107 |
| 6,456,872 | September 2002 | Y. Faisandier | 600/523 |
| 6,460,187 | October 2002 | M. R. Siegel | 2/114 |
| 6,476,581 | November 2002 | A. L. Lew | 320/107 |
| 6,532,379 | March 2003 | Stratbucker | 600/382 |
| 6,546,285 | April 2003 | Owen et al. | 607/5 |
| 6,551,252 | April 2003 | Sackner et al. | 600/536 |
| 6,560,473 | May 2003 | S. Dominguez | 600/382 |
| 6,561,814 | May 2003 | Tilbury et al. | 439/37 |
| 6,595,918 | July 2003 | Gopinathan et al. | 600/300 |
| 6,606,993 | August 2003 | Wiesmann | 128/204.23 |
| 6,647,552 | November 2003 | M. M. Hogan | 2/114 |
| 6,671,545 | December 2003 | R. W. Fincke | 607/5 |
| 6,681,003 | January 2004 | Linder et al. | 379.106.02 |
| 6,681,404 | January 2004 | Adlard et al. | 2/94 |
| 6,897,788 | January 2005 | Khair et al. | 340/870.16 |
| 6,930,608 | August 2005 | Grajales et al. | 340/573.5 |
| 6,973,673 | December 2005 | S. J. Beuk | 2/114 |
| 6,987,965 | January 2006 | Ng et al. | 455/419 |
| 7,020,508 | March 2006 | Stivoric et al. | 600/390 |
| 7,065,401 | June 2006 | M. Worden | 607/5 |
| 7,072,721 | July 2006 | C. Trent | 607/149 |
| 7,171,166 | January 2007 | Ng et al. | 455/73 |
| 7,197,357 | March 2007 | Istvan et al. | 600/509 |
| 7,261,690 | August 2007 | Teller et al. | 600/300 |
| 7,272,428 | September 2007 | Hopman et al. | 600/382 |
| 7,285,090 | October 2007 | Stivoric | 600/300 |
| 7,403,808 | July 2008 | Istvan et al. | 600/393 |
| 7,502,643 | March 2009 | Farringdon et al. | 600/509 |
| 7,532,931 | May 2009 | Gilkerson et al. | 607/30 |
| 7,539,536 | May 2009 | Schwartz et al. | 600/518 |
| 7,559,768 | July 2009 | Marmaropoulos et al. | 439/37 |
| 7,567,836 | July 2009 | Y. Zhang | 600/512 |
| 7,595,723 | September 2009 | Heitzmann et al. | 340/539.12 |
| 7,596,405 | September 2009 | Kurzweil et al. | 600/518 |
| 7,606,620 | October 2009 | Gilkerson et al. | 607/30 |
| 7,627,372 | December 2009 | Vaisnys et al. | 607/5 |
| 7,689,437 | March 2010 | Teller et al. | 705/2 |
| 7,715,905 | May 2010 | Kurzweil | 600/509 |
| 7,736,319 | June 2010 | Patangay et al. | 600/528 |
| 7,778,696 | August 2010 | A. Sathaye | 600/509 |
| 7,810,172 | October 2010 | J. D. Williams | 2/114 |
| 7,823,221 | November 2010 | J. S. Green | 2/114 |

### U.S. PATENTS IN APPLICATION

| | | |
|---|---|---|
| 2008/0184455 | August 2008 | P. W. Blume |

### FIELD OF SEARCH

This patent application is an invention in a fitted garment that would serve to provide life support to those people with heart problems when they are ambulatory in their community or at home. The fitted garment is designed to give a peace of mind to persons with histories of heart disease, and susceptibilities to a possible heart attack, or that have had an incidence of myocardial infarction that are all factors that can bring about a sudden cardiac arrest [SCA]. Sensors and defibrillation means are fitted in this garment so that it can monitor the person and their movements and activities. This fitted garment serves to provide warnings both to the person, to the cardiac technicians, emergency services, and their cardiac physician. The fitted garment gives life support directly to the cardiac patient by incorporating the latest technology in sensor means that send signals their cardiac physician and his staff directly if any unusual medical situation occurs. The fitted garment would contain defibrillation means that would be activated optimally within one minute of any medical situation. This quick response would automatically institute defibrillation of a person's heart, and by such means, avoid any untimely life threatening delays that often occur in a situation involving sudden cardiac arrest. Untimely delays in SCA of more than several minutes will result in severe brain loss of oxygen [hypoxia] and most often, in death.

The best class for this patent application seems in the clothing and garment classification in the CLASS 2: APPAREL, and this is covered in the sub-classification: 2/102. 113, & 114 wherein vests, wraps, shirts, and garments are shown. As this invention intends to give heart monitoring and possible lifesaving functions to the otherwise drab and ‘lifeless’ appearing hospital garments, and even to allow and encourage outside use from the hospital environment, and even then to add style and fashion for everyday uses. The primary support fitted garment can be used by itself such as a “tee-shirt”; however, when necessary for weather conditions and desired style, another shirt or blouse can be worn over the fitted garment without any problem.

As this patent application is for a fitted garment serving cardiac patients and those persons with a proclivity toward cardiac issues, and as such no claim is made upon any recent cardiac technology or sensor means to monitor these people, but rather to make use of the best suited devices that the primary cardiac doctor selects for this person.

The primary classification for the lifesaving function is found in the medical field in CLASS 600: SURGERY wherein the activities dealing with the human heart are the dominant factor. In this search the sub-classes most relevant are /382 to/396 with the highest impact seen in /388 “ . . . Garment” and /389 “ . . . Vest”, and an often cited reference in /393 “Plural electrodes carried on single support”, and then in the sub-classes /500 to /528 wherein /509 “Detecting heartbeat electric signal”, and /518 “ . . . Tachycardia or fibrillation detected”, and /528 “ . . . Detecting heart sound”, all seem important in this invention application.

An earlier classification is in the CLASS 128: SURGERY wherein the earlier patents reside, and the sub-classes most active are: /2.06; /639; & /644 wherein the same medical issues exist as above in CL. 600.

Another related classification is in CLASS 607: SURGERY, LIGHT, THERMAL AND ELECTRICAL APPLICATION wherein the activities of my invention seem most active in the monitoring and signaling of heart functions and disturbances. In this search the sub-classes are /4 “ . . . Combined cardioverting/defibrillating”, and in /30 “Remotely changing, [e.g., programming, pacer] parameters or operation”.

The activities of the means of wireless communication that transmit the data of a patient is seemingly continuously changing and upgrading its means. The means available to alert the primary care doctor or the medical staff or medical facility seemingly can be by radio transmission, by internet and satellite means, and by cell phone means when enabled. These means should involve the electro-cardio-graphic {ECG] signals as a heart rhythm gathered by the patient worn module device transmitted by the electronic modem to its base station, and thereupon to its computer processing unit [CPU, or in some cases directly by the module means from the patient's fitted garment. Although the means available is responsive to the distances involved, in all cases of any serious emergency, including SCA, it is timeliness that is in reality the most critical factor as more than 2 or 3 minutes [6 is the maximum] is all the time that the person will have for any realistic recovery to life. In a serious heart attack situation, only quick treatment would save heart muscle and brain hypoxia to nourish and keep alive the delicate tissues.

Some involvement and consideration therefore is accorded CLASS 340: COMMUNICATIONS: ELECTRICAL, wherein the applicable sub-classes are /539 “ . . . Radio”, in /539.12 “ . . . Medical”, in /539.13 “ . . . Tracking location” [e.g., GPS etc.], in /539.14 “Including remote residential device”; and references in subs /870.01 “Continuously variable indicating [e.g., telemetry], and in /870.16 “Condition responsive”.

Finally, in the CLASS 379: TELEPHONE COMMUNICATIONS, one sees the remaining means to send out data and signals commonly used today, in the cell phones or by transfer means to the wired telephones, in the sub-classes /106.02 “ . . . Patient monitoring”; and by cross reference to be seen in the regular CLASS 439 [also a telephone classification] in sub 439/37; and in the CLASS 455: TELECOMMUNICATIONS [a similar classification], in the sub-class /73 “Transmitter & receiver at some station [e.g., transceiver]”. These means offer a modem download of ECG to a hospital and their cardiology facility and the results of echocardiography that shows the value of the left ventricular ejection fraction [LVEF] by image & percentage remaining operable.

In the field of communications today the wireless uses of the ‘cell phone’ supplants many older hard wired telephone systems, and the ‘single use function’ of a cell type phone can send out alerts and the warnings that are typically transmitted from a ‘docking’ station or ‘base unit’. Many of these newer phone uses are incorporated herein that improve the communication for the patient and their heart monitoring so vital to their continued state of good health. As such many of these newer adaptive functions and phone uses will be described in this application for a patent such as is seen in the “Holter” monitoring in the transmission of ECG data & ultrasound imaging to the cardiac physician.

It is evident in current technology that in the use of cellular telephones can utilize in combination voice recognition with logical synthesis, and subsequently extrapolate text into speech. This software can recognize and interpret commands and requests being made, and can evaluate and follow conversations between a doctor and a patient. The software contains artificial intelligence from its stored data and patient information to answer basic patient questions and requests for services. This artificial intelligence can gather its data and interpret the routine actions of a doctor in response to a patient's needs. Such procedures in a cell phone can draw upon its data base from all sources to manage and interact across multiple interacting service requests. These procedures are benefits for the attending cardiac physician in prescribing medications when best serving the patient's needs and conditions. With a wealth of data and interpreted procedures the attending physician can consider the need for a cardiac pacemaker or another internal cardiac device implant [ICD], and even the more serious medical procedures to be evaluated such as coronary artery bypass grafts, balloon angioplasty and its possible additional drug exuding stent placement, or a balloon counter pulsation means, or possibly a left ventricular assist device placement, among others. Thus, in the procedures of modern cardio vascular management and care, this cell phone data means can prove a real benefit for the attending cardiac physician.

Of note however, the patent being applied for is for a fitted garment, and as such no claim is to be made for the invention of any telemetry or such improvements as may from time to time be enacted. This patent fitted garment is adaptable for such telemetry and such related technology as is now current, or will be developed in the future.

### BACKGROUND OF THE INVENTION

In the background for this invention application there is evidence of early concerns for the patient's dignity, and improved means to facilitate a medical examination, and support means for medical devices such as catheters, cell phone ultrasound, and other items. In this invention application it is the Apparel classification that holds the prime position to serve these medical garment concerns, and to offer an attractive appearance as well as serving its medical functions. In this application I offer a garment pleasing in style and color and function that is vibrant with “life” and not a somewhat typically drab hospital garment that by its very appearance seemingly “lifeless”.

In Class 2: Apparel, these early concerns using a garment to transport objects is seen in U.S. Pat. No. 2,156,504 issued May 2, 1939 to Bess S. Liss entitled “Acoustic Device Supporter” wherein a radio receiver and battery and antenna reception is provided. In my garment application the means to place such items can be available if desired, as the means and method is shown being provided to place such items within a single, softer, wearable undergarment that has two layers, of which the inner layer serves to carry the monitoring sensors, the transponder module and its power source, the defibrillation devices and the outer layer serves to conceal any such items, and it is the outer covering that gives vibrant color and style to the garment.

In the U.S. Pat. No. 2,688,752 issued Sep. 14, 1954 to Dominic G. Sbarra and Caroline M. Sbarra entitled: “Undergarment with Attached Article Carrying Harness” wherein is shown a strapping and cloth harness framework positioned upon the upper portion of the torso designed to “stitched or otherwise secured to an undergarment, such as a T-shirt, so that it is automatically put on and taken off with the undergarment; which includes a pocket for receiving a small object, such as a hearing aid unit; which is comfortable to wear and inconspicuous when worn beneath an outer garment; which is elastic and adjustable to eliminate the possibility of discomfort in its use; which supports the hearing aid unit at a location for the most efficient reception; and which is simple and durable in construction, economical to manufacture, effective and efficient in use, and neat and attractive in appearance.” [col. 1, lines 9-22] In my invention application any attaching devices would be within the softer layer of the undergarment, and not separately and externally placed, and then designed so that an outside layer of the undergarment be fashioned sufficiently strong enough to serve as a concealing layer for any such attaching devices, and then the combined layers can be conjoined into a single undergarment that can be worn alone just as a T-shirt would be, or it can be integrated with a vibrant and stylish outer shirt or blouse if weather or fashion dictates.

In the garment classification are herein cited two U.S. Patents as examples incorporating elements to protect and give security to infants. The first is U.S. Pat. No. 2,704,070 issued Mar. 15, 1955 to Annie B. Rudisill entitled “Non-Slip Baby Band” wherein in a vest type garment the lower section has non-slip material front and back that is designed to ameliorate slipping or falling of an infant. What seems germane to my application is the inclusion of material that can be equated to a device as included in a vest garment. What is different would be the single garment with its features and elements visible wherein in my invention such features and elements are not visible and not shown, being screened by concealment means so that when need be, the undergarment can function as a single shirt or blouse.

The second example chosen is in U.S. Pat. No. 2,918,677 issued Dec. 29, 1959 to Charles Pindyck entitled “Undershirts for Infants” wherein a single swift pull will open up the front of the undershirt making it easier to change diapers as an example. The single swift pull is in part enabled by conjoined snap fasteners variously held together by a woven tape to which the snap fasteners are attached, and thus enabling the single pull to open the garment. What seems germane to my invention is partly its ease of use, and partly the concealed structure holding the snap fasteners. What is different is the facilitating in my invention to hold and conceal medical devices, including even a cell phone when used as a medical device, and their containment and situation wherein they do not show visibly as the undershirt would have a layer of a screening material outside of any such devices or elements so that if desired the undergarment would serve as an attractive and wearable shirt or blouse.

In the U.S. Pat. No. 2,479,246 issued Aug. 16, 1949 to Beatrice Lupo entitled “Hospital Bed Jacket” wherein a dignity bed jacket is shown that allows selected front and neck opening for a doctor in an examination. Inventor Lupo's patent is selected as a good standard approach to a better type of bed jacket wherein in my application I have included other functions and elements allowing the placement and inclusion of medical devices and a type of garment that is not restricted to be used a hospital bed, but that can be worn on the outside of a hospital just the same as ordinary street clothes.

Another hospital dignity garment is seen in the U.S. Pat. No. 6,647,552 issued Nov. 18, 2005 to Mary M. Hogan entitled “Medical Dignity Garment” and assigned to Guided Inspirations, Inc. of Morris, Ill. [Inventor Hogan's town of residence] wherein are provided many openings and fold away flaps to facilitate a medical examination or procedure being performed without having to remove the garment. Inventor Hogan states: “The medical dignity garments of the present invention are manufactured to facilitate easy on and off of the natural appendages. The present invention garments are designed to support a healthy, life-giving image for people and to provide modesty to the level desired personally and culturally.” [col. 2, lines 52-56] What is evident is that inventor Hogan is performing many of the same functions and features of my invention application, except for the numerous opening examination flaps and the overall function of an outer garment whereas my invention will show a different application of the softer undergarment with its concealing means for medical devices and monitoring sensors hidden in a more durable outer layer both in a single garment being adapted to being used as a T-shirt, or more fashionable in term of a ‘polo’ shirt, or ‘golf’ shirt, or ‘beachwear’ shirt. Whereas inventor Hogan's very specialized outer wear is to facilitate medical examinations in a medical setting, my invention is different as it is for use long after the examinations are over, and it provides a convenience of walk-about medical monitoring wearing the medical devices in a fashionable and street usable garment to be worn outside of a hospital or medical setting, and for everyday use without any compromise to fashion or style.

These three prior art patents concern medical treatment issues that are different, but present a similar design and element that can be addressed to follow:

| | | | |
|---|---|---|---|
| 5,799,330 | Sep. 1, 1998 | Christine O'Donoghue-Kitt | 2/114 |

“Medical Treatment Garment”
A garment intended solely for treatment procedures, with openings and
flaps available for such.

| | | | |
|---|---|---|---|
| 7,823,221 | Nov. 2, 2010 | Jacqueline S. Green | 2/114 |

“Garments for Holding Post-Surgical Drain System”
A specialized gown shown with two drains held in pockets from a double
mastectomy surgery operation.

| | | | |
|---|---|---|---|
| 2008/0184455 | Aug. 7, 2008 | Patricia W. Blume | 2/114 |

“Gowns for Cardiac Patients and Other Specialty Uses”
A hospital type gown is shown with post surgery care means for monitors,
catheters, tubing, drainage, small instruments in two front pockets.

In the invention application to follow many differences are to be seen from the treatment garment inventor O'Donoghue-Kitt as above, and the hospital use gowns of inventors Green and Blume. Differences including the fact that in my invention the patient or citizen can wear my garment in public view, wash it easily, and wear it for a long time, all the while it does still serve its intended function for as long as it is needed.

In the prior art patents, these seven show tubing and drainage post surgery, and several medical procedure garments that show garments covering the upper body of medical patients typically while in a hospital setting. These patents are prior art, but they do not deliver the features and elements that are included in this invention application. None of these seven patents indicate the garment is wearable outside of a hospital setting, as this application will show its adaptability and long term use and means to be worn on the street and in the public view.

These seven are listed to follow:

| | | | |
|---|---|---|---|
| 6,460,187 | Oct. 8, 2002 | Marilyn R. Siegel | 2/114 |

“Medical Clothing”

“Multipurpose medical clothing, such as a gown or robe, that safely accommodates medical appliances attached to a patient, affords privacy, and thus encourages the patient to be up and ambulatory.” [Abstract, lines 1-4]] The garment will manage sensors, telemetry, catheters and the requisite tubing, and IV delivery, as required. [source: Abstract] Inventor Siegel's medical clothing is intended for supportive use within the hospital, and not as in this application, street use and being in the public outside of the hospital.

| | | | |
|---|---|---|---|
| 4,582,508 | Apr. 15, 1986 | Wilma F. Pavelka | 604/179 |

“Garment for Receiving Catheters and the Like”

This garment is a strapping device designed to hold catheters in use, and it is not a true garment such as a T-shirt. Inventor Pavelka's device is designed to serve rather specific needs, and it is not a garment to be worn in the public view as it is in my invention.

| | | | |
|---|---|---|---|
| 5,806,096 | Sep. 15, 1998 | Jacqueline R. Pennington | 2/80 |

“Medical-Tube Retaining Garment”

This invention is well explained in its Abstract, and to quote therefrom: “A medical tube-retaining garment to deter patients, especially children, from disturbing as surgical wound site on the torso. An inner garment resembling a shirt or undergarment having a split back is affixed to an outer garment made of a wrappable, elastic bandage material. Both the outer and inner garments close over the back by overlapping portions having hook and loop fasteners, which rear entry design deters the patient or child from detaching the garment.” [source: Abstract] My invention is not oriented to tubing, and is not for wound issues, and is not designed as rear opening, among other differences.

| | | | |
|---|---|---|---|
| 6,973,673 | Dec. 13, 2005 | Staci J. Beuk | 2/114 |

“Intrusive Device-Supporting Apparel”

This invention demonstrates great concern for IV attachments in detailing the attaching strips of strong fabric that would hold and position the tubing so used. A major difference from my invention would include the use of inventor Beuk's apparel being used and worn in a hospital setting, and not being intended as public clothing.

| | | | |
|---|---|---|---|
| 7,810,172 | Oct. 12, 2010 | Judy D. Williams | 2/114 |

“Garment for Accommodating Medical Devices”

This invention shows a large front panel in the garment wherein the panel is removable to ‘provide access to device sites’, and additionally serves with a hidden pocket inside for ‘absorbing or deodorizing material’. Structural supports are available to hold ‘monitor wires, tubing, or other devices’.[source: Abstract] In my invention application the garment would be designed to be worn in public view, whereas inventor Williams intends her garment for hospital uses only.

| | | |
|---|---|---|
| 5,708,978 | Anna C. Johnsrud | 2/102 |

“Medical Vest”

As stated in the opening sentence: “This invention relates generally to patient care apparatus and more particularly to a patient garment for carrying a portable medical appliance.” [col. 1, lines 1-3] Further is stated: “The present invention provides a carrier for portable modules such as infusion pumps, chemical injectors, heart monitors and the like . . . ” [col. 2, lines 32-34] While inventor Johnsrud does incorporate additional batteries and their weight as well, and she restates that an infusion pump is the primary purpose, it is evident that her garment is not to be worn in public view and has no means to conceal any medical appliances such as is evident in my invention.

There are now ten prior art patents with physiological elements shown in the garments that need review herein as they relate to my invention application:

These first three show electric means to perform better exercise or sports, to stimulate and improve muscles to exercise with, and give better posture.

| | | | |
|---|---|---|---|
| 7,072,721 | Jul. 4, 2006 | Cecillio Trent | 607/149 |

“Electrode Vest for Electrical Stimulation of the Abdomen and Back”

| | | | |
|---|---|---|---|
| 6,551,252 | Apr. 22, 2003 | Marvin A. Sackner & Dana | 600/536 |
| | | Michael Inman | |

“Systems & Methods for Ambulatory Monitoring of Physiological Signs”

| | | | |
|---|---|---|---|
| 4,889,131 | Dec. 26, 1989 | Robert J. Salem & George H. | 128/671 |
| | | Holley | |

“Portable Belt Monitor of Physiological Functions & Sensors Therefor”

The next three by Granek et al. indicate 18 electrical points to use both to deliver an electrical pulse, and also, to receive as sensors input of the selected areas.

| | | | |
|---|---|---|---|
| 4,729,377 | Mar. 8, 1988 | Herman & Murry Granek, & | 128/639 |
| | | John Church | |

“Garment Apparatus for Delivering or Receiving Electric Impulses”

| | | | |
|---|---|---|---|
| 4,583,547 | Apr. 22, 1986 | H. Granek et al. | 128/639 |

“Garment Apparartus for Delivering or Receiving Electric Impulses”

| | | | |
|---|---|---|---|
| 4,580,572 | Apr. 8, 1986 | H. Granek et al. | 128/639 |

“Garment Apparatus for Delivering or Receiving Electric Impulses”

The next two patents are: LaBove & Mann are directed to force feeding of someone with a high nutritional liquid By IV means; and the Ricketts et al. patent shows a means to place electrodes movably upon a belt and monitor physiological functions of a person.

| | | | |
|---|---|---|---|
| 4,087,864 | May 9, 1978 | Larry D. LaBove & Patrick A. | 2/102 |
| | | Mann | |

“Dispensing Vest for Patients Receiving Hyperalimentation”

| | | | |
|---|---|---|---|
| 4,026,278 | May 31, 1977 | James R. Ricketts, Corrine M. | 128/2.06 |
| | | Ballantine, & Gerald J. Reiser | |

“Electrode Positioning & Retaining Belt”

The last two patents in the physiological list with different inventors shown are both assigned and filed by Lifecor, Inc. of Pittsburg, Pa., and each has similar functions with shaped electrical pulses being administered to a patient for selective and treatable diagnosis.

| | | | |
|---|---|---|---|
| 6,280,461 | Aug. 28, 2001 | Glegyak et al. | 607/5 |

“Patient-Worn Energy Delivery Apparatus”

| | | | |
|---|---|---|---|
| 6,065,154 | May 23, 2000 | Hulings et al. | 2/102 |

“Support Garment for Patient-Worn Energy Delivery Apparatus”

In these several prior art patents to follow the uses of pockets, pouches, belts, and small holders even for softer materials is to be listed.

| | | | |
|---|---|---|---|
| 1,092,144 | Apr. 7, 1914 | S. Grushlaw | [apparel class] |

“Pocket for Sweaters”

| | | | |
|---|---|---|---|
| 4,411,267 | Oct. 25, 1983 | A. M. Heyman | 128/385 |

“Telemetry Transmitter Holder”

| | | | |
|---|---|---|---|
| 4,666,432 | May 19, 1987 | K. McNeish et al | 604/174 |

“Catheter Retaining Means & Method”

| | | | |
|---|---|---|---|
| 6,561,814 | May 13, 2003 | Tilbury et al. | 439/37 |

“Garment Carrying Electronic Devices”

| | | | |
|---|---|---|---|
| 6,681,404 | Jan. 27, 2004 | T. K. Adlard et al | 2/94 |

“Garment with Pouch for Medical Monitor”

In these several prior art patents to follow, there is seen belts and supporting garments.

| | | | |
|---|---|---|---|
| 3,409,007 | Nov. 5, 1968 | E. W. Fuller | 128/2.06 |

Body Electrode Support Garment”

| | | | |
|---|---|---|---|
| 3,534,727 | Oct. 20, 1970 | J, A, Roman | 128/2.06 |

“Biomedical Electrode Arrangement”

| | | | |
|---|---|---|---|
| 4,498,480 | Feb. 12, 1985 | J. L. Mortensen | 128/644 |

“Adjustable Probe Belt Assembly”

| | | | |
|---|---|---|---|
| 4,608,987 | Sep. 2, 1986 | H. E. Mills | 128/639 |

“Apparatus for Transmitting Data”

These several prior art patents to follow become more specific as to being able to offer patient-worn cardiac monitoring devices, and as such become more useful as relevant art for this invention. As these prior art patents offer selective features, elements, and functions that are useful or in application use for my invention to follow.

| | | | |
|---|---|---|---|
| 4,698,848 | Oct. 13, 1987 | M. C. Buckley | 2/114 |

“Blouse for Cardiac Patients”

A comfortable and attractive garment with front closing “designed for use in all cardiac rehabilitation situations including exercise and stress testing” [Abstract, lines 5-6] One major difference is the placement of monitor electrodes directly upon the skin by adhesive means which is not done in my patent application, and another is the front closing requiring a belt.

| | | | |
|---|---|---|---|
| 5,611,085 | Mar. 18, 1997 | V. Rasmussen | 2/102 |

“Garment for Holding an Electrocardiographic
Monitoring Unit and Cables”

This patent shows “current registration of electrocardiographic measurements or similar measurements on a person wearing the garment by a portable monitoring unit, with wires and cables connected to electrodes adapted to be secured in contact with the skin of the wearer . . . ” Abstract, lines 1-5] What is different is the lack of style in a basic type garment, and again that sensors are directly upon the skin by adhesive means unlike my invention.

| | | | |
|---|---|---|---|
| 6,930,608 | Aug. 16, 2005 | L. Grajales et al. | 340/573.5 |

“Apparel having Multiple Alternative Sensors &
Corresponding Method”

This invention in its opening Technical Field cites: “This invention relates generally to human and/or environmental monitoring and more particularly to such sensors as carried about by a person.” [col. 1, lines 7-9] The sensors are mostly physiological and in part cardiac capable, and although portable in the garment, and monitored by radio transmitter and supporting network, the intent is a complete means to monitor the physiological condition of the wearer and transmit some of the data received to be stored. The term apparel, or items of apparel, “refers generally to all manner of clothing and corresponding accessories, such as but not limited to shirts, coats and jackets, vests, pants, shorts, socks and gloves, shoes and boots, underclothing, and hats, helmets, and other headgear [col. 3, lines 28-34; and in claim 3] and yet in my invention the apparel is one garment as in a T-shirt. The sensor activity is shown with its cardiac activity in FIG. 8; and in claim 5, but dominantly the concern is a broad range of general physiological activity. There seems little means to give needed data for heart patients and support through pacing, alerts, and no indication of defibrillation in an emergency such as is shown in my invention.

Now in the prior art one comes to the field of technology and its inclusion for cardiac situations. In my invention application herein it is the fitted garment with its means and methods using a two layer undergarment as the cardiac monitoring base that provides life support by intelligent means such as pacing the heart beat to help control atrial fibrillation and low energy defibrillation prior to the emergency situation of sudden cardiac arrest requiring a means to administer full power defibrillation. There seems to be many improvements in the technical means to help support heart patients, and such devices are continuously being upgraded. In the patent application herein such upgrades and improved technology is easily incorporated into the fitted garment.

Selections of any technology and cardiac support means would basically be the domain and the decision of the primary doctor in charge of cardiology. The doctor will decide the best means and method for their patient, and the medical facility and staff will support the patient 24/7/365 always with the best technological means chosen by the doctor. Selections herein are examples of the prior art and representative of the art that is in use or available. In the design of the fitted garment all such examples can be used as chosen by the primary doctor in charge.

Evidence in the current technology shows monitoring electrodes being free of direct skin contact [see U.S. Pat. No.6,930,608, col. 2, lines 54-55], of these electrodes being wirelessly connected to the pickup module [see patents to follow], and to be able to transmit continuously data to a docking station, and computer processing unit, and even today into a cell phone telemetry system. Placing any such heart monitoring sensors, or the defibrillating means, in a fitted garment would be easy to accomplish, and as such, would fit into my invention application as herein submitted.

The four patents to follow are examples of physiological sensors that give data and activity indicators, but are not monitoring the heart functions to serve as life support in pacing or defibrillation.

| | | | |
|---|---|---|---|
| 7,689,437 | Mar. 30, 2010 | E. Teller et al. | 705/2 |

“System for Monitoring Health, Wellness and Fitness”

| | | | |
|---|---|---|---|
| 7,502,643 | Mar. 10, 2009 | J. Farringdon et al. | 600/509 |

“Method & Apparatus for Measuring Heart Related Parameters”

| | | | |
|---|---|---|---|
| 6,606,993 | Aug. 19, 2009 | W. P. Wisemann et al. | 128/204.23 |

“Integrated Physiologic Sensor System”

| | | | |
|---|---|---|---|
| 6,595,918 | Jul. 22, 2003 | G. Gopinathan et al. | 600/300 |

“Tele-Diagnostic Device”

The following four patents have to do with gathering data from monitoring sensors on a heart patient with wireless means and data storage means with a computer processing unit. Also, beneficial is the evidence that the primary heart doctor and his staff, and the trained technicians on duty 24/7/365, and they can evaluate the situation and respond by return means to the patient, and then initiate and implement life support and emergency care to this patient. As such these patents add great value to this application for the fitted garment as they show the usefulness and importance of today's technology to give life support and even life saving to a heart patient.

| | | | |
|---|---|---|---|
| 7,715,905 | May 11, 2010 | R. C. Kurzweil et al. | 600/509 |

“Cooperative Processing with Mobile Monitoring
Device & Computer System”

| | | | |
|---|---|---|---|
| 7,596,405 | Sep. 29, 2009 | R. C. Kurzweil et al. | 600/518 |

“Atrial Fibrillation Detection”

| | | | |
|---|---|---|---|
| 6,681,003 | Jan. 20, 2003 | M. Linder et al. | 379/106.02 |

“Data Collection & System Management for Patient-Worn
Medical Devices”

| | | | |
|---|---|---|---|
| 5,224,479 | Jul. 6, 1993 | Y. Sekine | 128/644 |

“ECG Diagnostic Pad”

The next seven patents as examples have to do with wireless sensor means, and/or wireless telemetry, or transmitting means that eventually can reach the primary doctor or his staff, or the 24/7/365 technicians on duty. As this application is for a fitted garment that can serve as a means to contain these sensors, and as such, it can support the data gathered, or any life saving activities deemed necessary. What is important here is that all of these monitoring systems can easily be placed into the fitted garment when selected by the doctor in charge and the supporting medical staff.

| | | | |
|---|---|---|---|
| 7,595,723 | Sep. 29, 2009 | H. A. Heitzmann et al. | 340/539.12 |

“Wireless Communication Protocol for a Medical Sensor System”

| | | | |
|---|---|---|---|
| 7,403,808 | Jul. 22, 2008 | R. Istvan et al. | 600/393 |

“Wireless ECG System”

| | | | |
|---|---|---|---|
| 7,272,428 | Sep. 18, 2007 | N. C. Hopman | 600/382 |

“Wireless Electrocardiograph System & Method”

| | | | |
|---|---|---|---|
| 7,197,357 | Mar. 27, 2007 | R. Istvan et al. | 600/509 |

“Wireless ECG System”

| | | | |
|---|---|---|---|
| 7,171,166 | Jan. 30, 2007 | R. Ng et al. | 455/73 |

“Programmable Wireless Electrode System for Medical Monitoring”

| | | | |
|---|---|---|---|
| 6,987,965 | Jan. 17, 2006 | R. Ng et al. | 455/419 |

“Programmable Wireless Electrode System for Medical Monitoring”

| | | | |
|---|---|---|---|
| 6,897,788 | May 24, 2005 | M. Khair et al. | 340/870.16 |

“Wireless System Protocol for Telemetry Monitoring”

| | | | |
|---|---|---|---|
| 6,295,466 | Sep. 25, 2001 | A. Ishikawa et al. | 600/509 |

“Wireless EKG”

| | | | |
|---|---|---|---|
| 6,289,238 | Sep. 11, 2001 | M. Besson et al. | 600/509 |

“Wireless Medical Diagnosis & Monitoring Equipment”

The final three examples have to do with electronic means to monitor, to pace, and to regulate the heart rhythm and some use a low power means of defibrillation that is attempted prior to the sudden cardiac arrest mode that requires the full voltage for the defibrillation to be called upon. My fitted garment can accommodate all such means and equipment, and all types of defibrillation pads either wet types or the newer dry pads- and with its outer layer serving to conceal all of these devices to offer a fashionable and stylish garment that can be worn in the public view.

| | | | |
|---|---|---|---|
| 7,627,372 | Dec. 1, 2009 | G. A. Vaisnys et al. | 607/5 |

“System & Method for Presenting Defibrillator Status
Information While in a Standby Mode”

| | | | |
|---|---|---|---|
| 7,065,401 | Jun. 20, 2006 | M. Worden | 607.5 |

“Method of Applying Electrical Signals to a Patient
and Automatic Wearable External Defibrillator”

| | | | |
|---|---|---|---|
| 6,671,545 | Dec. 30, 2003 | R. W. Finke | 607.5 |

“Defibrillator with Controller Operating in a Low Power Mode”

As is evident, the fitted garment would best serve persons with known heart disease and should be the prescriptive of choice following a heart attack of Myocardial Infarction [MI], and even before such an event if the person is known to have the issues and conditions that can lead up to a heart attack. Thus, anyone wanting a peace of mind would want the fitted garment to be prescribed for security and safety. In such cases the prescription for the fitted garment could well alleviate the risks for a Sudden Cardiac Arrest [SCA].

In preparing this Non Provisional Application I have reviewed and printed in part some 200 patents, and herein I have selected the better examples of the many available to give my invention a firm, solid base in the prior art. I have opened up the prior art in the Background of the Invention with apparel and garment items both somewhat in general and then somewhat medical in their function, and such is to show that my invention does fit in this classification and is unique and non-obvious to those of the prior art and those skilled in such apparel. I will continue now to show how and why my fitted garment should secure its letters patent.

### SUMMARY OF THE INVENTION

This invention application is for a fitted garment comprising the means of heart monitoring with defibrillation capabilities. This garment is intended to serve the needs of ‘at-risk’ cardiac patients to perhaps save their life; and to offer to anyone susceptible to heart problems these same advantages. The fitted garment is designed to give a peace of mind to persons with histories of heart disease, and susceptibilities to a possible heart attack, or that have had a recent incidence of a heart attack or myocardial infarction. All of these heart conditions are factors that can bring about a sudden cardiac arrest [SCA] that today is 95% fatal. In a SCA outside the hospital oxygen does not get to vital organs, including the brain, the heart, the lungs, the liver, and other essential organs, and the person quickly dies. Survival of someone in a SCA situation after 10 minutes is nil, and only at less than 5 minutes is any reliable survival realistic.

Although the fitted garment is designed and equipped to handle SCA in an optimal survival manner, it seems that early on coronary disease is evident and known long prior to any SCA, so that heart disease is where the fitted garment could be the most effective in saving a person's life. Coronary [heart] disease can be detected before such a disaster would occur and this is where this invention of the fitted garment can prove invaluable. It monitors the heart rhythm of the wearer to yield data indicative of an impending stroke or heart attack. A heart attack is different from a SCA, as in a conventional stroke or heart attack the blood flow is slowed and does not move well, but oxygen is still in the blood, and it does get to the brain, the heart, and other vital organs. SCA is now usually thought of as a terminal event. The means within this fitted garment to monitor the ECG and gather the heart's data is a life saver for the person with coronary disease which is an early indicator for strokes, heart attacks, and SCA. Abnormal heart rhythms [arrhythmias] occur in more than 90% of the people who have had a heart attack. Proper use of the fitted garment can be instrumental in the control of heart attacks, of strokes, and the prevention of any SCA. The fitted garment can be a life saver and would give a peace of mind to the wearer.

This fitted garment can offer a successful and satisfactory monitoring of a person's heart rhythms long before any serious coronary disease and emergency situation would occur. This fitted garment is a comfortable, easy to wash and clean, and a pleasant and fashionable means to dress, plus any heart monitoring would be unobtrusive in this garment system. The early detection and successful management of a coronary situation would be gained from the use of this fitted garment, as any debilitating SCA would typically be indicated by such early monitoring. Consider that there is a graduated risk of SCA such that early monitoring would indicate as there is an accumulation of factors and indicators of a patient's heart disease. Such measurable heart disease issues can include the ‘Left Ventricular Systolic Dysfunction’, Congestive Heart Failure, a previous heart attack, Ventricular Tachycardia that is sustained or out of rhythm. Patients with an “Ejection Fraction” [EF] from Zero to 30% [aka weak pumping of the Left Ventricle] are recognized as having a very high incidence of SCA. As their EF improves indicating that there is some healing, the incidence of SCA does improve in that situation.

This fitted garment can be instrumental in the early detection and in the management of heart disease that can grow into a serious condition. In the current state of heart disease there are guidelines to be observed in implanting an ‘internal cardiac defibrillator’ in a person at risk for SCA that is most often fatal, and this fitted garment again can represent a life saver for this person from the accumulated data being received from the continuous heart monitoring. For example, there is a 3 month waiting period to see if the person improves the LVEF activity. It seems that the fitted garment can be very important in knowledgeable decisions as the longer that the person survives with heart disease during even these most critical situations, the better are their chances of living past a SCA situation, and returning to a normal pattern of life. The data provided so easily and so completely by the fitted garment will yield a record of a person's heart rhythms to ascertain whether an implanted defibrillator would be necessary. In fact, in recent medical studies it has been observed that a reduced LVEF whether by natural mending of the body over time, or by medication support, or an implanted defibrillator control and activation, it has been established that the reduced LVEF is clearly the most important determinant for overall mortality in SCA and its risk of death. It can be realized that this invention can effectively lead to a reduction in mortality from SCA that is now considered almost universally a terminal event.

Another strong consideration for this invention is the support that it can give to patients with heart disease and myocardial infarction [MI] wherein the home use of an Automatic External Defibrillator [AED] would reduce the all-cause mortality. The AED availability and use is for patients with a history of anterior MI and heart distress indicators. These patients are not candidates for an ICD and thus were assigned an AED that in the event of a SCA, their available support group or family members perform Emergency Medical Services [EMS] by the means of Cardio Pulmonary Resuscitation [CPR] and/or the use of an AED to resuscitate the stricken family member.

Yet, another strong consideration for this invention is that the indications in the medical observations show that relying on bystander intervention and defibrillation is inadequate and unlikely, even when the bystander has been trained in the use of an AED. This fitted garment comprising heart monitoring and comprising defibrillation is a much better answer than relying on bystander's intervention when needed. It needs to be said that the AED is still needed and very useful, especially with young people and those with no previous history of heart disease, and they suffer a SCA situation. And while, this invention of a fitted garment can serve to ameliorate such situations, there is no claim made for such functions with regard to medical aspects and the means to determine the onset of medical conditions.

Additionally, this invention will offer to anyone wearing this garment, a designer fashion with style and color that will be vibrant with ‘life’. Nothing would seem to be worse than to give a person suffering under such heart conditions, a somewhat typically drab garment that by its very appearance offers a seemingly ‘lifeless’ garment. As we proceed through this invention application, the medical advantages and security offered will be understood, together with a fitted garment that can go outdoors, and into the public eye, and go shopping normally, and to be attractive with fashion and color showing a vibrant life.

A fitted garment by its definition could comprise several ways to achieve its purpose, and in the embodiments to follow selected applications will be offered. In order to achieve its exterior attractiveness and style, this invention can comprise an outer garment with these characteristics that will meet the public eye, so to speak, and yet, a garment that will contain its useful features supporting the medical devices and power means so necessary to serve its medical purposes. The fitted garment will comprise the means of heart monitoring comprising the means for multiple sensors to detect the heart rhythms. Then, additionally fitted, as an undergarment or supporting framework, the multiple sensors that would be fitted close to the skin of the person for the necessary heart monitoring means with defibrillation capabilities, if needed. These garments are integral in close combination to provide the full service of medical features as herein outlined.

Garments can comprise the several separable elements such as the fitted undergarment that is by a tight fitting means tight on the wearer's skin to gain the best possible register of the heartbeat rhythms for the monitoring process, and this undergarment would also contain the defibrillation means tight to the skin, if the need arises. A severe heart situation wherein abnormal heart rhythms, such as ventricular tachycardia or ventricular fibrillation, for example, would indicate the need of an emergency heart defibrillation, then immediately a response within one minute of 150 to 200 joules of electric shock would be administered to assure the person's survival. A power means can include capacitors to administer the electric shock and typically might be mounted in a radial fashion around the defibrillator pad in an expeditious manner to deliver their power as requisite. In such a cited manner, the defibrillator can be wireless with an antenna available to receive the command from its controlling module. In another manner the defibrillator can be a dry mounting covering the pad with a selectively electrically conductive membrane that remains dry to the touch, and yet functional to a high degree. Thus, a ‘dry’ type defibrillator could remain in the fitted garment day after day, and used more than once, without any problem, unlike the wet type that can really be used only once and must be replaced after each use. And, thus, the fitted garment can offer peace of mind to the wearer.

This undergarment serves to provide the prime monitoring means that is so necessary for the wearer and for this invention to serve its highest function. This undergarment can provide its functions additionally by strapping means if such would be more satisfactory to achieve the high level of sensor monitoring of the wearer's heartbeat or ECG, so that a shock can be administered in the case of ventricular tachycardia and/or ventricular fibrillation, and for pacing of the heart rhythms in the case of ‘asystole’ of the heart. This undergarment would have containment means for each individual heart sensor, and for the several defibrillation pads required, and the containment means for an availability of medicines, such as aspirin, beta-blockers, inhibiters, diuretics, and specific cardiac drugs and syringes. There are over 1 million persons each year in the US. that experience a heart attack, and this fitted garment can offer a satisfactory means of survival for many persons who would otherwise die. The heart attack can be confirmed by their ECG, or ultrasound test, and their cardiac support network would be alerted of this emergency through the data received from the wearer borne electronic module carried in the fitted garment. Half of the deaths from a heart attack occur in the first 3 or 4 hours after the symptoms begin. This invention of a fitted garment could be used to confirm these symptoms as a medical emergency, and as such, the fitted garment can indeed be a life saver, so that this person can be taken to the hospital promptly. The sooner that treatment of a heart attack begins, the better is survival and a return to a life that is routine for the fitted garment wearer. Chewing an aspirin tablet after an ambulance has been called can help reduce the size of the blood clot causing the heart attack. Oftentimes another useful intermediary means can be a beta blocker—if available—that can slow the heart rate, thus lowering its oxygen needs. The aspirin and other medicine aids can be worn in the containment means provided [oftentimes a pocket] in the fitted garment so that these aids are easily accessible to the wearer of this garment. The person's awareness of the need for an early treatment can be instrumental in saving their life, and this invention of the fitted garment can be that instrument. If the person survives the heart attack without medical intervention, after 6 hours, most heart damage would be permanent. This fitted garment would give the required medical alert to the cardiologist and hospital even if the person wearing the garment was asleep or unable to respond. This invention can be a real time life saver.

This garment invention could thus have several garment elements to achieve the highest and best means to support a targeted heart patient, or any person so inclined to want the protection and safety afforded by this invention. Garment elements can comprise the undergarment, the straps if required, the separable outer garment with style and fashion, and in selective situations a shielding garment means for intrusive medical devices or telemetry means of the module and antennae. Thus, garments can comprise separable outer garments with containment means and separable fitted undergarments with containment means comprising integral heart monitoring means with defibrillation means and ultrasound testing means. Garments further comprising separable garments with containment means for multiple heart sensors with defibrillation means provide heart monitoring of the heartbeats to a wearer borne module. Garments can further comprise that the wearer borne module comprise the means to transmit and receive its data externally. In most embodiments the separable outer garment would provide the containment means to support the weight and carry the module and its power supply, and the containment provision should be ergonomically provided by supporting means up in the garment near to the shoulder. The higher placement would be easier to support and less interference or motion found at the beltline.

The fitted garment comprising the multiple sensors that comprise the heart monitoring of the heart rhythm will pass electronically to a wearer borne module that will in turn accumulate its data, evaluate, and store or transmit its data externally to another computer processing unit [CPU]. The fitted garment would comprise multiple sensors that comprise the means to deliver the heart monitoring of the heart rhythm and comprise wireless means. In a preferred embodiment, the heart monitoring by sensor means would be wireless in that each selected electronic sensor, would provide data to an electronic module that is capable to accumulate said data from each sensor, and that module can be carried upon the person, even possibly by a cell phone means. The fitted garment comprises that the wearer borne module may be fitted by containment means with in said garment. The fitted garment comprises the containment means comprise the power means for the wearer borne module to transmit and receive its data externally. The garment therefore would comprise a fitted undergarment with containment means as requisite for medical devices, and further comprise a separable outer garment with containment means. This garment thus would comprise the means for the outer garment with containment means and the fitted undergarment with containment means be integral in containment for the requisite medical devices.

The necessary elements that support these sensors and the module such as the power means and the transmitter and receiver means can be carried in an outer garment designed ergonomically to be capable of supporting the weight comfortably, yet in a concealing way. The advantage of such an outer garment shell can be its removal for washing or dry cleaning, and its changeability with other outer stylish garments as the need arises. The feature of being able to change this garment shell is important to the person wearing it, and the exchanging and securing of the medical devices, including the packaging of medicines and syringes available for heart attacks, can be accomplished by the use of pockets, pouches, or compartments tastefully concealed in the garment. The adaptability of the medical devices when wireless can be served by linking them together in a sequence to yield the optimal heart monitoring; or if in a wired sequence, by linking them together by the use of specialty plugs that uncouple each individual monitor until relocated in another garment. Securing each device from falling out of the garment can be by hook and loop means, and these means can include fold over pockets flaps, or other preferred interior securing means.

As a person's data including—hopefully—a normal heart rhythm, is delivered to the module that is carried with the wearer of the garment, this small electronic device will transmit and receive information from its command processor that can link up to the emergency and cardiology centers for support and instructions as the need may arise. The fitted garment comprises the means to alert the wearer's cardiology facilities of the cardiac situation when the heartbeat is irregular. The command processor unit [CPU] can interpret the wearer's heart signals and evaluate the heart functions and situation from its connection to the cardiology data base and its preloaded experience data, so that it can manage the heart monitoring, and it can issue changes or alerts or warnings as the need may arise. The typical CPU would be able to record the wearer module in its routine functions, and transmit out to another CPU, or cardiology facility so that in an emergency situations, the alert and the need for help can be issued timely. The fitted garment further comprises the wearer borne module continuously transmit and receive data of the wearer's cardiac situation in such an emergency. In the situation wherein the electronic module perceives that a SCA event is indicated, and the person is awake or mobile and hears the loud and distinctive tone indicating a defibrillation shock is immanent, but unnecessary, then a manual override can be activated that forestalls the defibrillation shock. If the person is asleep, or unable to respond, the SCA defibrillation shock would be automatically administered unless forestalled by the cardiologist or emergency department of the hospital.

The capabilities of these CPU's to transmit and receive data are enormous, and as such, they are capable of monitoring heart rhythms, and they can detect any abnormalities with great precision, and they can automatically issue commands for any changing or stressful situation, including defibrillation. In terms of the numbers, the sudden cardiac arrest [SCA] is the number 1 killer in the United States with currently over 450,000 deaths, and as such, far more deadly than strokes, lung cancer, breast cancer, or auto immune deficiency syndrome [AIDS] that are the current next highest numbers of deaths when all are combined. It seems evident that few survive this SCA event, and at present, it is estimated only about 5 percent outside the hospital will survive when discovered timely and treated. It is known that when these persons are treated with defibrillation with an AED within 3 minutes of the SCA event, that up to 74% will survive. Thus, a self-testing, full service electronic monitoring means with defibrillation capabilities offers great hope and great success for persons with heart damage, incipient family history of heart disease, cardiomyopathy indicators, Congestive Heart Failure [CHF], or a prior heart attack. This invention serves these means to offer a safe, secure and comfortable fitted garment that can contain and support any and all of the best electronic systems available that can save many persons from heart attack death, or the SCA and fatality. The fitted garment can be worth every penny spent.

The fitted garment further comprises the means of the wearer borne module to receive data to command said sensors to attempt to achieve the regulation of the heartbeat by a pacing means to return to a routine heartbeat. Additionally, the fitted garment comprises an adjustable power means comprising the means to initiate defibrillation means in the event of a sudden cardiac arrest to restart the wearer's heart. This garment worn by a person or heart patient would then be a lifeline to health and security when coupled to such enormous capabilities and competence.

With this garment and its features, the heart patient can be reasonably mobile, and safe when wearing this garment. The newer technology for transmitting and receiving signals is continuously expanding its range and its abilities to provide optimal coverage for anyone using this garment. More powerful CPU's and module systems, and better procedures to control heart monitoring, and better ways to administer life saving techniques evolve over time to offer the wearer of this garment invention security and comfort of use. While this fitted garment as an undergarment monitors the heart sounds and can provide upon instruction, the pacing and the defibrillation procedures as the need arises, the outer garment shell covers these devices with style and fashionable upbeat colors, and supports the fitted undergarment as the carrier of the power means and oftentimes the module means, so that together they are a team that provides the security and comfort for this invention. An observation in the ‘Journal of the American Medical Association’ [JAMA] notes that “The risk of sudden death is early on. The longer you survive, the better you do.” This fitted garment with its working inner undergarment and its integral supporting outer garment is exactly what the doctor ordered to prevent a SCA death.

This level of security and comfort that is provided by this fitted garment invention can serve for hospital uses, and as well, residential care that can include the eldercare and assisted living facilities, and as needed, mobile management that can be just as safe. The wearer of this fitted garment can even document their ‘LVEF %’ over time by means of a module recording ultrasound. These are excellent features that can extend life and give comfort to heart damaged patients, and provide a higher level of their quality of life when under such stressful circumstances. This fitted garment invention extends security and comfort to many persons not hospitalized, or targeted for intensive care, that are able to walk around while being monitored.

In the catastrophic event of sudden cardiac arrest that is the ultimate threat to any person with heart irregularities, the cardiac care system that is installed in the fitted garment has the means and ability to automatically offer the means of defibrillation to restart and pace the heart promptly and expeditiously. When a patient or person has been fitted for this garment usage, their heart conditions would soon be known and any irregularity would be detected and sent to be stored and evaluated in the connecting CPU system. The fitted garment would be sending out signals for the normal, and for any irregular, heartbeats. Quickly, the attention of a cardiologist, or of a medical care facility, would be involved, and all concerned would be alerted to impending cardiac difficulties so that close monitoring and supervision would be instituted, and emergency care could be administered if the need arises. As time is extremely critical in a sudden cardiac arrest situation, every second counts. The immediate attention of someone available and trained sufficiently for the activity of a defibrillation situation can offer a successful procedure to save a life. Experience has shown that five minutes seems the maximum to have a chance of success, and three minutes is optimal for recovery, and so in most situations an expected procedure can be programmed into the cardiac response system of the fitted garment. An automatic defibrillation can be undertaken within the one or two minutes that can assure a high level of success. Additionally, since the alert would be automatic of an irregular heartbeat, and an ancillary cardiac failure, it can be programmed into the CPU and the wearer's module to begin first attempting to restore normal heart function by induced pacing measures promptly, as the electric shock strength can be adjustable as needed. Indeed, this measure can itself be life saving in the case of the heart arrhythmia that is known as ‘asystole’. The measure of promptness of actions in these cardiac situations would make all the difference in success for a fitted garment wearer.

In most conventional situations with a sudden cardiac arrest, few persons can receive proper medical procedures within the first five minutes that would give the person a 50/50 chance to regain a normal life, and to forestall their death. In many situations the available outside party is untrained and unfamiliar of the method and procedures required to restart a person's heart by defibrillation means. Again in these situations, the available party is uncertain, and unfortunately possibly unwilling, to risk such a hands-on procedure that is required to administer defibrillation from an outside device, and again, possibly afraid of any legal ramifications from trying to save a life, but likely failing. In this invention the fitted garment would have been preprogrammed for such a situation, and would be automatic, and would require no bystander's intervention even if there is the typical emergency device of an AED [the Automatic External Defibrillator unit], and such a unit can be quickly located, and properly set-up, and by the instructions properly administered. Simply stated, the fitted garment would handle such a situation better, increasing survival from the current 5% to upwards of hopefully 90%. As this is also supported by a cardiac facility competent to supervise these procedures already having monitored the person's heart condition, and the fitted garment would have the available medicines and devices for a heart attack, or stroke, in a garment containment pocket, and it would have the means of defibrillation installed in its electronic module to initiate restarting a person's heart, as the need arises. This invention would additionally have the means to continue to monitor the person's heart activity and condition closely so as to be ready if further defibrillation or pacing of the heart is deemed necessary. Such follow up procedures and monitoring are not normally available without this fitted garment invention. In the usual condition of a heart attack, or a sudden cardiac arrest event, the individual would be unable to institute a defibrillation procedure on their own, and the cooperation of some attending outside person would be required promptly within the critical two or three minutes to begin the defibrillation procedure even if such an AED device is available. The advantage of this fitted garment invention is obvious, and it offers a life saving feature not usually available by any other means. It seems also obvious that in most situations the person so affected by the sudden cardiac arrest would die.

In this invention the introduction of a full-care fitted garment could forestall many life threatening situations. This fitted garment in its combinations will contain the needed medicines and syringes conveniently located in a pocket, and it can operate its medical devices automatically, so that it does offer a peace of mind not generally available to heart damaged patients, even those having had a prior heart attack. While the fitted garment will provide a measure of style and comfort for these heart patients, it is truly the electronic age that brings the features of this invention together to provide safety and security to the heart damaged patient. It is the fitted garment in its undergarment and outer garment configurations that gives this assurance, and the mobility far beyond normal to these patients and others that today's usual methods cannot give. In emergency situations this fitted garment can react quickly through its electronic means to any irregular heartbeat to offer recovery, and normal rhythm pacing, and if the need arises, a defibrillation procedure. The reaction to any of these heart difficulties is quick and decisive, and it is guided by the cardiologist and the medical cardiology center. In such situations it has proven beyond the capability or willingness of outside parties to take charge and try to save a person's life Where time is of the essence, measured in seconds, and not minutes, the automatic features provided in this invention are exemplary as heart saving procedures would be in seconds and within one to one and one-half minutes which provides a good and solid safety margin for the person with the heart difficulties. Electronics and its technology will continuously upgrade over time, as will the flexibility, distance, and range of the electronic module to serve the fitted garment wearer. This combination of the fitted garment will continuously offer better and better means and features to improve a patient's or a person's life with heart disease.

### In a Best Embodiment

It is the object of this invention to provide the means for a garment comprising the means to be fitted on a person comprising heart monitoring with defibrillation means. The garment further comprises that the fitted undergarment provide the means for holding cardiac medicines that treat heart attacks, and the attachment of the heart monitoring means comprising defibrillation means. The garment further comprises the heart monitoring means comprising defibrillation means comprising multiple sensors and pads attachable to the fitted undergarment. The security and safety of the wearer is thus provided by the objects stated, and the tight and proper fitment of the undergarment that contains the sensors and pads that give this service, and a manual override means that allows the wearer to cancel, or forestall, a defibrillation shock when alerted by a distinctive and loud tone of an immanent defibrillation. In the cases when irregular heart palpitations can create a false triggering of an immanent defibrillation shock, either the automatic override from the module will control or cancel this event, or the manual override—if provided—can cancel this event.

In providing the fitted garment services, it can be seen that several elements and applications would be requisite. The critical undergarment so adjustable to be tight and properly fitted to the wearer's skin so as to be able to receive the heartbeat signals that support life. Integral to this fitted undergarment would be the outer garment that in turn supports the weight of the power supply and the module if requisite. The outer garment would be a designer quality shirt or blouse with style and color and fashion that would allow the wearer to enjoy all normal activities and mobility. In selective situations, a shielding effect might require an intermediate garment, or portion of a garment, to shield or disguise intrusive medical devices or antennae, so that the package of garments would be able to maintain their purposes and support of the heart monitoring securely and safely. Additionally, the basic garment would comprise the means for the outer garment with containment means and the fitted undergarment with containment means to be separable and washable as needed.

In the numerous selections of the outer garments that identify style and fashion and a vibrant quality of life, perhaps, two or more per package, and the required tight fitting undergarments, perhaps two or more per package, it can be realized that the patient or person wearing these garments would select, perhaps, two or more packages, to wear. The electronic module, a new battery power source, the garment pocket with a container for the medicines and syringes for treating heart attacks, and such supporting equipment that would be needed, would require only one supply per installation, but the garment selections can be numerous.

This invention of the fitted garment would prove flexible over time as upgrades or newer features can be incorporated in its design and functions. While it is obvious that sensor means and defibrillator means will change over time, there would be no difficulty with adapting any such upgrade and newer features over time. An example of such flexibility can be seen with the extensible uses in the newer technologies being available and being developed in cell phone uses. The fitted garment would easily adapt to provide garment containment means for such newer cell phone uses and any ancillary requirements in their uses and services. These cell phones may well serve as an ancillary module with upgraded ultrasound features, including echocardiography and Doppler flow measurement features that allow non-invasive viewing of the person's beating heart. The provision for a cell phone means being incorporated in a fitted garment can allow anyone to “see” into the beating heart, even for viewing a fetus, and for providing enough detail in the heart chambers for discrete viewing just as though it were being lighted internally. Echocardiography can lead to measurement of a left ventricular ejection fraction [LVEF] as a measurable percentage of its beating strength, as well as many necessary medical observations. These observations can include fluid in the pericardium, congenital heart diseases, blood clots or tumors in the heart [that can break off causing stroke], active infection in the heart valves, abnormal elevation of pressure within the lungs, and other medically important issues.

ECG analysis from sensor electrodes in the fitted garment can reveal nerve conduction problems within the heart such as a bundle branch block, and T-wave functions. Such stress test results can be very revealing and helpful in medical diagnosis, and available instantly by the means of a cell phone ancillary module. Any cell phone owner with a weak heart that has these features available through his cell phone will appreciate their importance in understanding of receiving this data, and the importance of understanding the occurrence and meaning of their heart palpitations, and how this cell phone can help with medical diagnosis and corrective treatment. The fitted garment as described herein can show the ease and the importance of such services to anyone with heart disease and the issues there from.

The electronic services and the medication formulary would be at the behest of the cardiologist, their registered nurses, or at the cardiology center of a medical facility, all that would write the prescriptions for medicines, and as such would be individually selecting for their patients the best suited from the many sensor and defibrillation devices available. Since these doctors and medical centers would be attending the wearer of the fitted garment routinely and in emergency situations, they would best be able to select the electronic components and medicines best suited to the wearer. Additionally, these medical doctors and professionals would best be able to supervise the installation of sensors and pads contained in the fitted undergarment, and they would oversee the transmitting and receiving of the ECG signals from the wearer's heartbeats. In any emergency the doctors and staff would have the capability and training to deal with any irregularity in the wearer's heart rhythm, and be in control either manually, or by the automatic means preprogrammed for any individual patient or situation.

### BRIEF DESCRIPTION OF THE DRAWINGS

FIG. 1 is PRIOR ART. A frontal view of a person with defibrillator wet gel pads requiring assistance of someone nearby to pull out the attached cords that allow the gel in the pad to come out for the electric shock to be administered effectively; and a signal device on the belt with the switch means to operate the defibrillation pads to initiate the defibrillation shock means to restart the person's heart in the event of a SCA event. Cardiopulmonary resuscitation [CPR] is often used because of time delay in administering a defibrillation shock.

This procedure is electric and now out-of-date.

FIG. 2 is another similar frontal view of a person shown dressed in a dress shirt that would constitute the outer garment that covers the undergarment as seen in FIG. 4.

FIG. 3 shows a soft shirt as an outer garment similar to FIG. 3 preceding. The outer garment covers the undergarment that provides the heart monitoring and defibrillation technology.

FIG. 4 shows the undergarment that is the fitted garment that is integral with the more concealing outer garment. This undergarment shows wireless heart monitoring sensors in place and with the wireless defibrillation pads, being available if the need arises, and would have the containment means [not shown] for medicines and syringes to treat heart attacks.

FIG. 5 shows the undergarment as a fitted garment with a wired system connecting the heart monitors, and the connected defibrillation pads, and the connecting wires passing up to the shoulder. What are not shown would be the connectors to the power source, and the electronic module that is needed to regulate and protect the person's heart, and the containment means for medicines and syringes to treat heart attacks.

FIG. 6 shows the integral outer garment for FIG. 5 that serves to conceal the wired connectivity of the heart sensors and defibrillation pads of the undergarment.

FIG. 7 shows an inside front view of a fitted garment as the undergarment that shows the straps as fitment means to tighten this undergarment so as to receive satisfactory signals from the heart monitors and maintain a tight fit so that the defibrillation pads can satisfactorily deliver the required shock, if the need arises. The containment pocket is shown, if convenient inside, for medicines and syringes to treat heart attacks.

FIG. 8 shows a similar view, but in reverse as it is the inside rear view of the same garment as in FIG. 7. The fitment means can be seen available in the straps that are typically with hook and loop fastening means, so that this fitted garment can close tightly to receive satisfactory and measurable signals for heart monitoring and the means for the defibrillation technology to be activated.

FIG. 9 shows a front-on direct view of the undergarment that can be tightly fitted to the person so as to be able to monitor the heart signals, and to be able to administer the proper defibrillation shock, if the need arises. The containment pocket is also shown for medicines and syringe, if needed, to treat heart attacks.

FIG. 10 shows an undergarment with its attaching means for wireless connecting heart monitoring sensors, and for wireless defibrillation pads. This undergarment with its covering outer garment can offer style and fashion for a person to travel outside of a hospital environment, and yet this person would be safe and secure in the event of unusual heart beats or severe heart symptoms.

FIG. 11 shows a similar view to FIG. 10 wherein the heart monitoring and defibrillation devices are wired and showing a connector plug allowing detachment as needed. The electronic module and the power source in both FIGS. 10 and 11 are shown

FIG. 11 continues: to be outside of the fitted garment, and most typically would be carried upon the outer garment, or by the means of a sling device that would rest upon the person's shoulder, as such a placement would tend to relieve the weight and bulk of these items.

FIG. 12 shows an undergarment wherein the wiring means is shown as it is connected in the garment. These wires should be in a designation of ‘flat wires’ as such would tend not to show contours through the garment. These wired means would pass into an electronic module that both interprets and stores the person's heartbeat record and notes any irregular heart rhythms, and as such, can transmit and receive data, if any further help seems necessary. This module and the power source are intended to be stored and carried outside of the undergarment, and they would likely be in the outer garment, or by an outside sling means that rests upon the person's shoulder. An ancillary module can be included provided by a cell phone with the wireless means to transmit data, and receive electric energy signals to institute defibrillation of the heart [triggering the capacitance discharge].

FIG. 13 shows a frontal view of a person with the fitted undergarment that contains heart sensors that are wireless and with defibrillation pads that are wired and contain wet gel. In these heart monitoring and defibrillation means the containment means for any and all can include hook and loop, as well as, clips, pins, snaps, pockets [partial or full] and such that would serve to support the devices either individually, or by means of a framework of devices, that can serve to carry the weight and deliver a tight fitment that provides satisfactory signals for every device provided.

FIG. 14 shows a specialty device that is the front view of FIG. 15 to follow, and it shows a single monitoring and defibrillation [gel] device fitted properly to provide signals and connectivity to its inclusive electronic module so situated to give a clear view to the person of its activity. In this configuration the electronic module would be in clear view and accessible to the person, and fully automatic in heart pacing and shocking.

FIG. 15 shows a rear view of the specialty device of FIG. 14 above, and it shows a single monitoring device centered upon the back of the person, and this device carries both a wet gel defibrillation electrode and several monitoring sensors. Of note: this wet defibrillation device remains dry upon the skin until a signal is received to administer a defibrillation shock wherein by capacitive powered means the several wet gel capsules are compressed to exude gel upon the skin, but it can only be used one time and must be then replaced. The fitted undergarment would position and carry these specialty devices in a satisfactory manner to provide a comfortable and easy to wear garment without the current means showing straps and belts that support this technology. Additionally, with the integral outer garment the person so fitted can venture safely and securely into the public and go to restaurants and shopping. For both garments, an easy detachment is provided of the electric wiring and devices that would allow these garments to be cleaned and laundered.

The preferred means for a defibrillation pad would remain as a dry one that would remain in use more than once and remain dry to the skin using the shielding of an electrically conductive plastic membrane that delivers the appropriate shock in joules of energy, functional, yet dry. This dry method would be preferred by every wearer of a fitted garment.

### INDEX NUMBERS TO FIGURES

1 Prior Art
2 the Fitted Garment
3 the Fitted Garment as an undergarment
4 heart monitoring wired sensors
5 heart monitoring wireless sensors
6 defibrillation pads—dry
7 defibrillation pads—wet
8 defibrillation—wireless connectivity
9 defibrillation—wired connectivity
10 fitment straps
11 fitment straps—hook & loop
12 the outer garment
13 garment containment means
14 the Electronic Module
15 wireless connectivity
16 wired connectivity
17 flat wired connector
18 the Power source
19 Strapping means
20 Belting means
21 Antennae means

### DETAILED DESCRIPTION OF THE INVENTION AND DRAWINGS

This invention offers a fitted undergarment for the heart monitoring technology required and the defibrillation means if required. This invention combines an integral outer garment for everyday wear of style and fashion suitable for traveling, shopping, and eating out while being protected with the fitted garment and its technology.

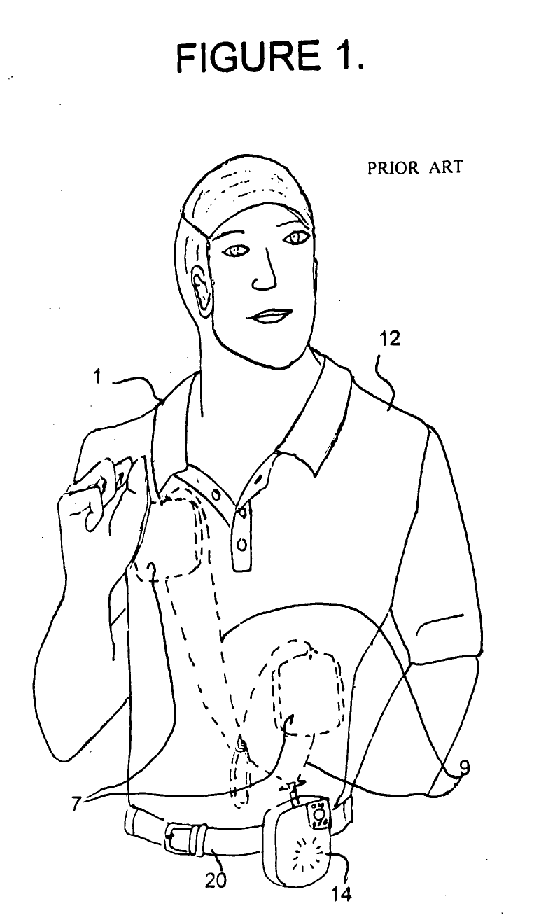

In FIG. 1 is seen the prior art [1] of the earlier days using manual pull cords shown in a loop above the belt that delivers the gel to the surface of the skin to facilitate the delivery of the electric shocks from the defibrillation pads [7] to a person's heart. A conventional outer shirt [12] is shown secured by a belt [20] that supports the electric switch [14] that equals an electronic module in current technology. In the prior art the regular applications are wired [9] for their connectivity in order to deliver the necessary energy for activation of the defibrillation pads [7] so that in the drawings to follow the upgrades in technology can be shown and detailed.

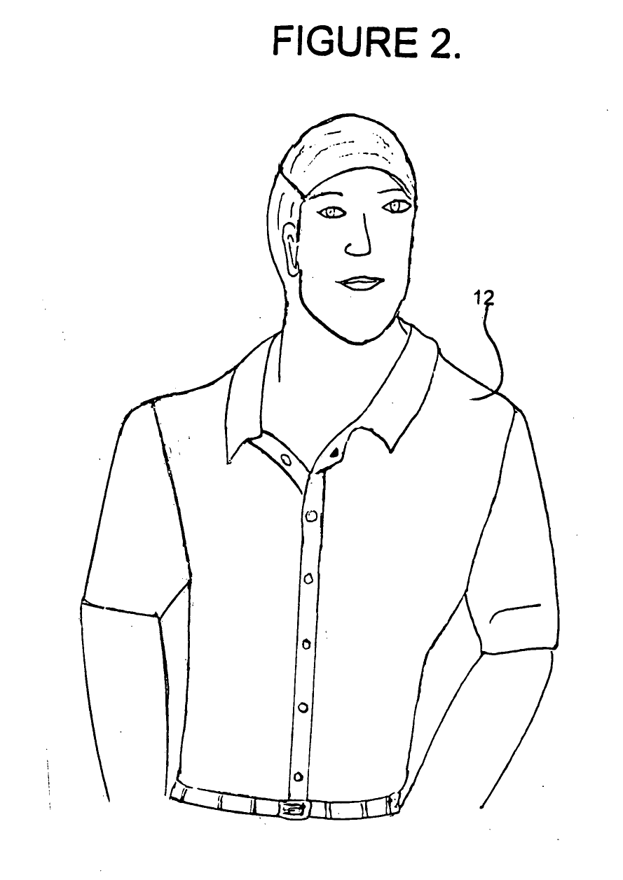

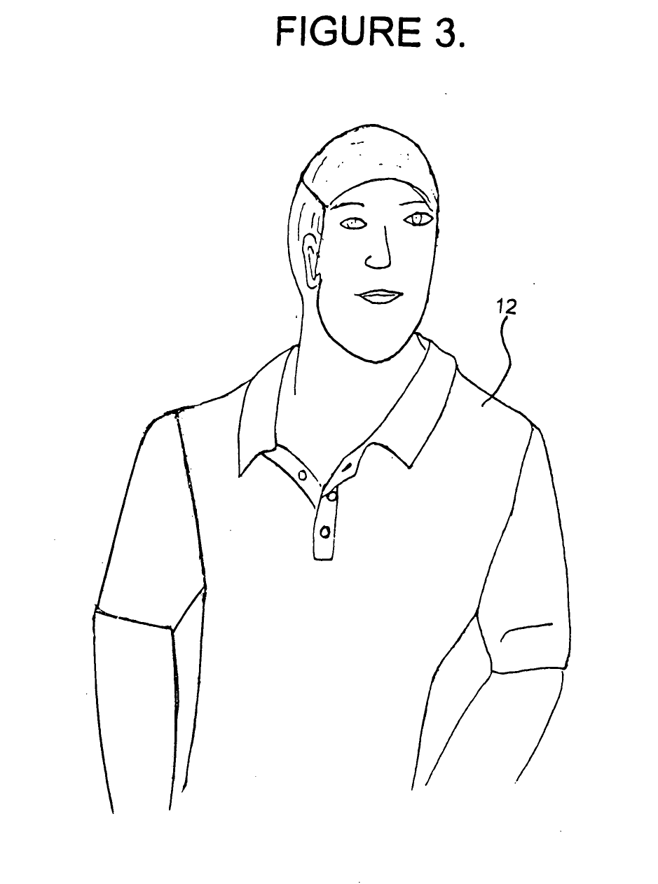

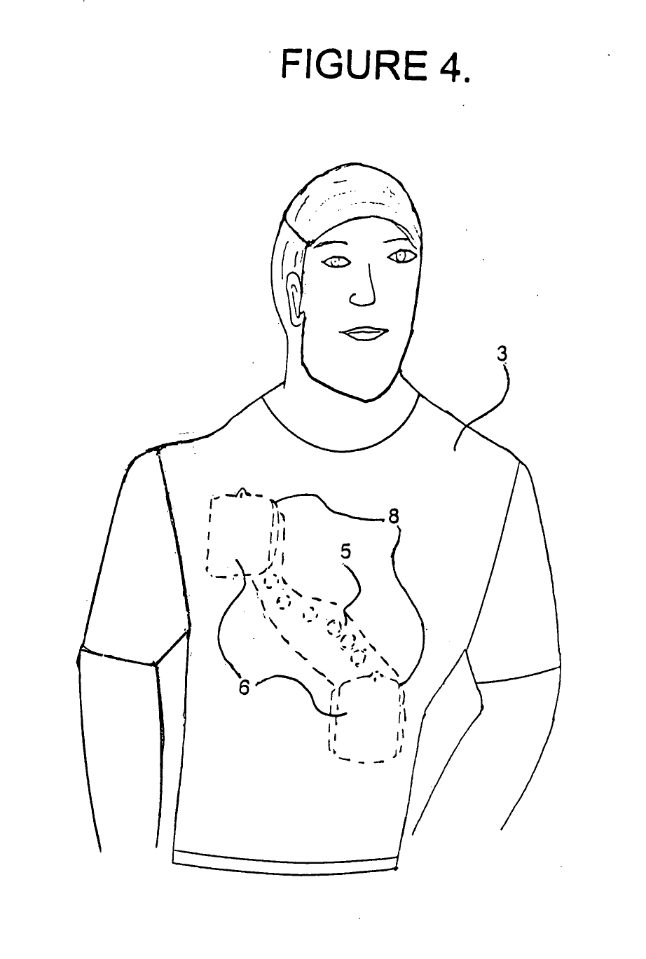

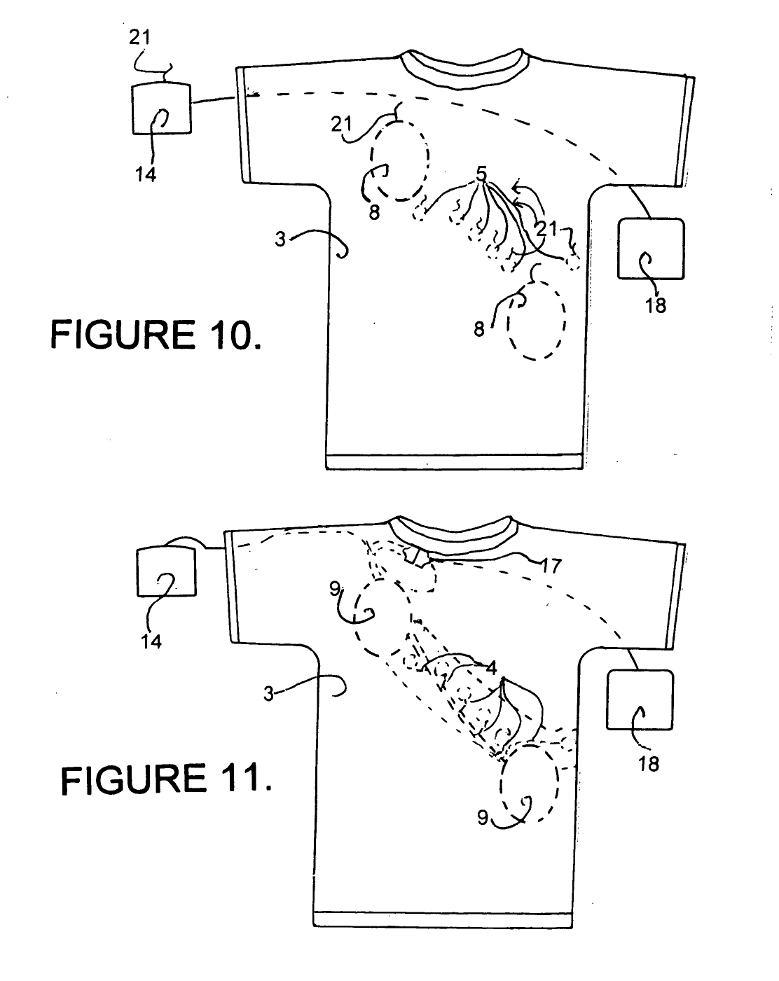

As this invention of the fitted garment [2] integrates the use of two garments to support the heart monitoring and defibrillation as the need arises, in the FIGS. 2 and 3 one sees the outer garment [12] that delivers style and fashion to cover the actual working inner garment. In the FIG. 4 to follow the working inner garment [3] is shown with wireless monitors [5] that transmit their data to an electronic module [shown [14] in FIGS. 10 through 15 to follow]; and wireless defibrillation pads [6] with antennae means [and capacitor discharge also by wireless means] that is a new technology. The fitted undergarment [3] is capable of supporting against the person's skin the heart monitors [5] and the defibrillation pads [6] so as to receive the best signals of the heart monitors and secure contacts of the defibrillation pads of any manufacturer and supplier of this equipment. The garment containment means [13] can be included in this fitted undergarment [not shown] either as a frontal side or as a backside pocket for the medicines and syringe to treat heart attacks

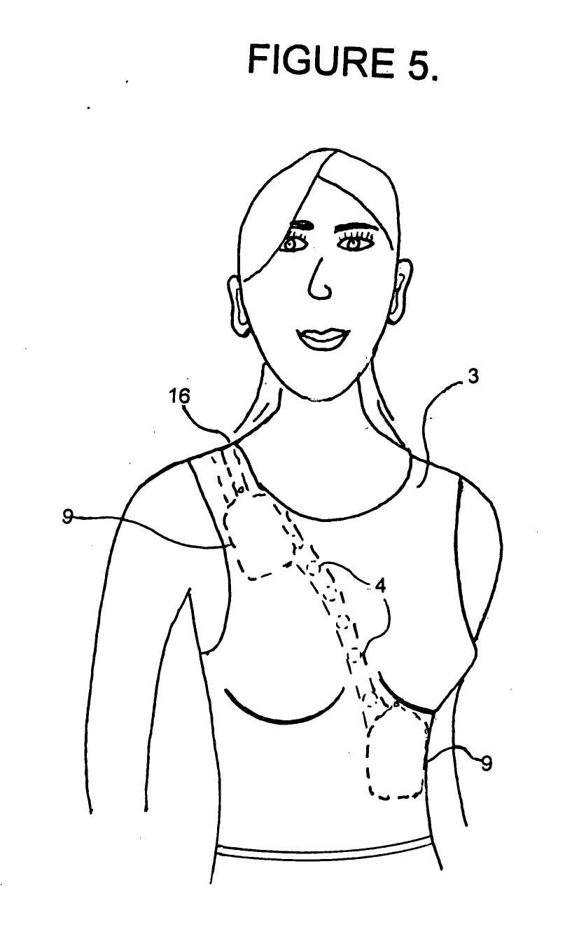

The next fitted undergarment [3] is in FIG. 5 that shows the wires [16] supporting the heart monitors [4] and the defibrillation pads [9] that give safety and security to people that feel the need for heart protection. This FIG. 5 does not show the flat connectors that join the power source [18] with the electronic module [14] and the garment containment means [13] of a pocket for medicines and syringes to treat heart attacks. The convenience of a medication formulary being available to treat heart attacks would be a tremendous benefit to everyone wearing the fitted garment.

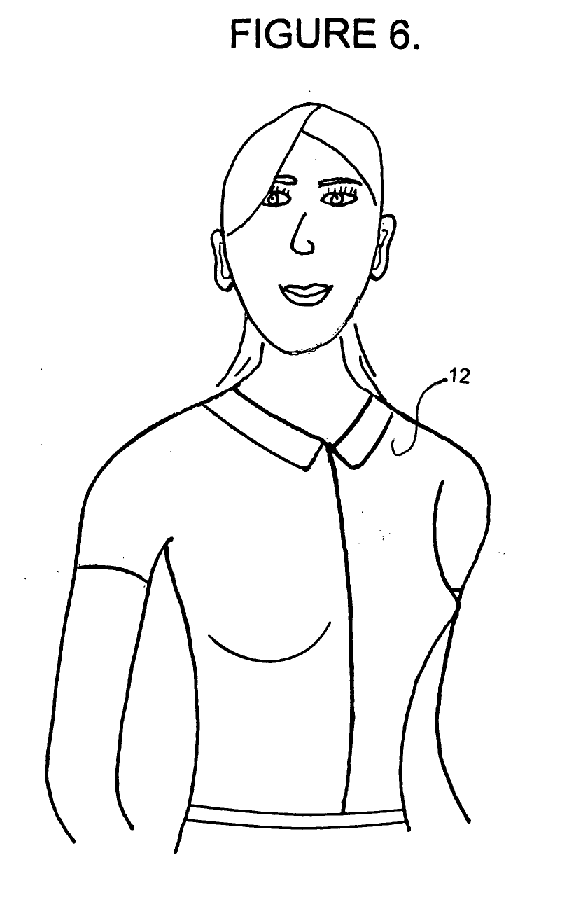

In the next view is FIG. 6 that shows the coverage of the person by an outer garment [12] that provides style and fashion and color to mask the serious business of the fitted undergarment [3]. In these fitted garments [of 2, 3 & 12] the packaging for a cardiac situation should include several of the fitted undergarments [3], possibly even three or more, and as well, several of the outer garments [12] that can be designer fashions for style and outside use in the public arena of restaurants, shopping, and travel. Another positive feature would be that they are washable, or can be dry cleaned if required, so the inclusion of several fitted garments [2] would be a welcome feature in packaging this invention.

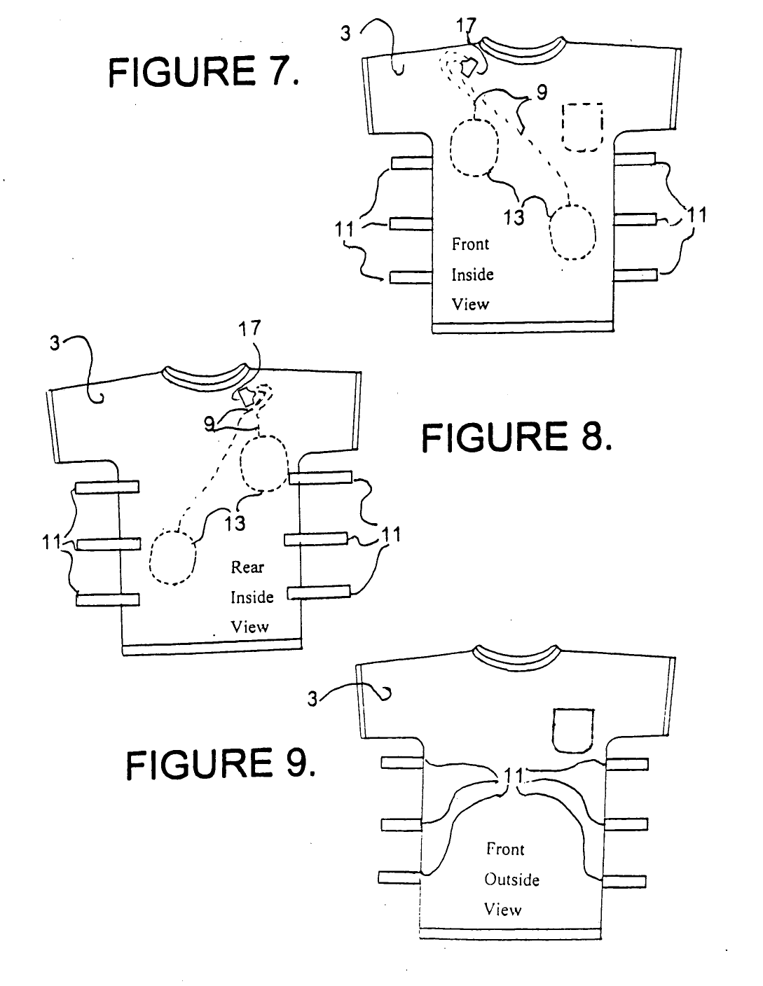

A set of drawings follows with FIGS. 7, 8, & 9 of the fitted undergarment [3] showing the fitment straps [11] of hook and loop means that are available to make the fitted undergarment [3] snug and tight to the person's skin for the best signals of the heart monitors [not shown], and for the best application for defibrillation pads located in garment containment means [13] and shown with wired connectivity [9]. In the FIG. 7, a garment containment means [13] is shown as an inside pocket holder for a specialized container for a medication formulary prescribed by the cardiologist to treat heart attacks. To follow in the FIG. 9 is shown the same medications formulary in a front pocket on the fitted undergarment [3]. In the FIGS. 7 & 8, a flat wired connector [17] is seen that would serve to connect to the control module [not shown] and power source [not shown] if needed. In the FIG. 9 only the plain exterior is shown for this fitted undergarment [3] and the connectivity to the electronic module and power source is to be seen in the FIGS. 10 and 11 to follow.

A wireless system for the fitted undergarment [3] is shown in FIG. 10 wherein six heart monitoring wireless sensors [5] show their wireless means antennae [21] that would send their heartbeats to the electronic module [14] with its antenna [21]. A power source [18] typically with long life batteries would supply the necessary electric current for the electronic module [14] to receive and transmit data, and the defibrillation means with wireless connectivity [8] to receive its command signal to initiate a defibrillation shock. In the wireless configuration the defibrillation shock would be administered by the means of capacitor discharges in order to provide the necessary joules of shocking power.

A wired system for the fitted undergarment [3] is shown in FIG. 11 wherein six heart monitoring wired sensors [4] are shown being connected to the wires that transmit their heartbeat signals to the electronic module [14] that is shown being outside of the fitted undergarment [3]. The power source [18] comprised usually of long life batteries is also shown outside of the fitted undergarment [3] as both the electronic module [14] and the power source [18] are intended to be carried in the integral outer garment, or possibly in a sling means under the front side of the shoulder, to alleviate the burden of uncomfortable weight. The flat wired connector [17] is a separable plug that can allow the electronic module [14] and the power source [18] to be disconnected when the fitted undergarment [3] is in place upon the person and the outer garment [12] is to be added to cover this fitted undergarment [3]. The two pads for defibrillation in wired connectivity [9] would be controlled by the data available to the electronic module [14] for a defibrillation shock if the need arises.

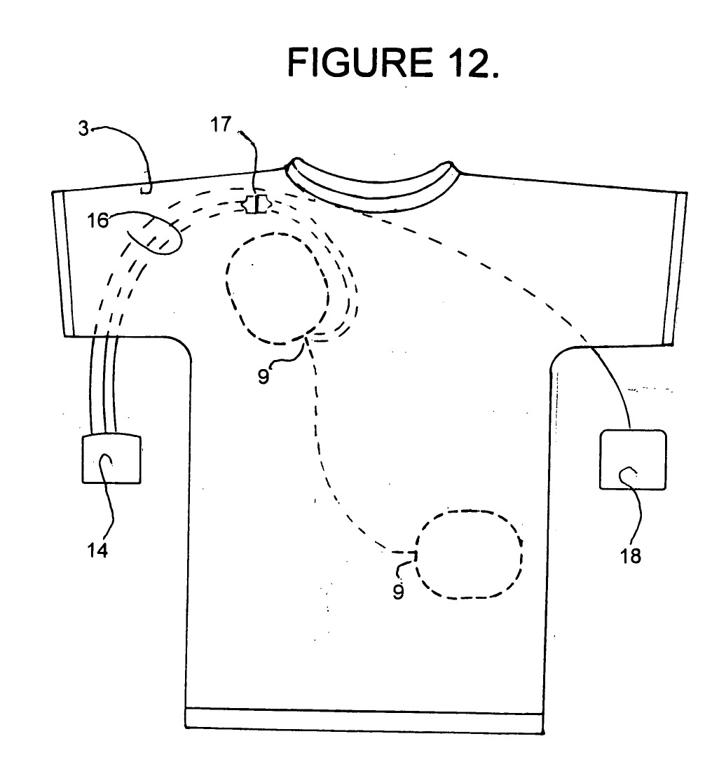

The FIG. 12 shows the fitted undergarment [3] wherein the two pads for defibrillation in wired connectivity [9] are shown without any heartbeat monitoring, and are shown connected to the external electronic module [14] that is similarly connected to the external power source [18] as detailed in the FIG. 11 above. This wired version shows the wired connectivity [16] means separable by a flat wire connector [17] that facilitates the placement of the external components in the integral outer garment [12] when it is used to cover the working fitted undergarment [3].

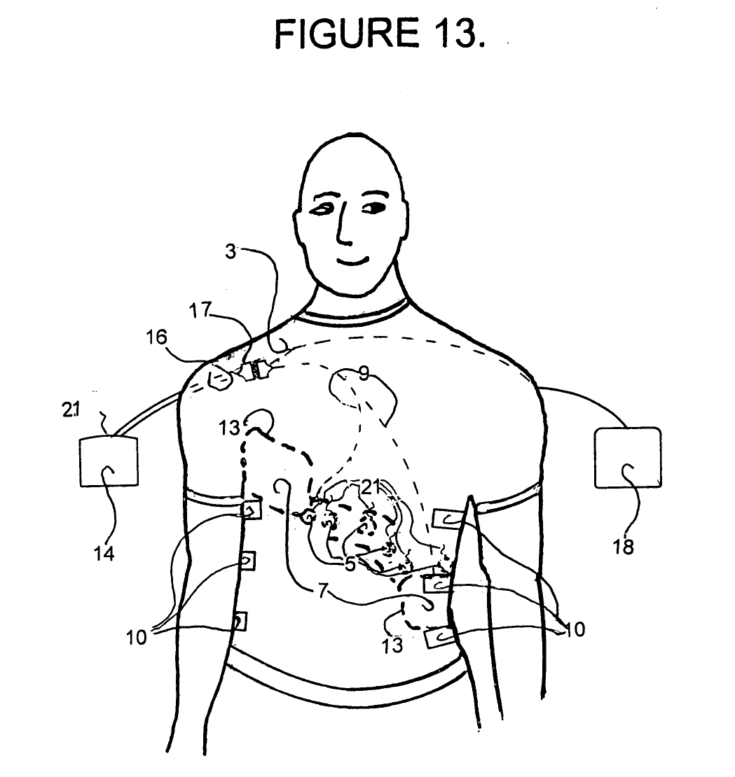

Another more complete model of this invention can be seen in the FIG. 13 wherein the fitted undergarment [3] shows the fitment straps [10] that serves to tighten the garment snugly to the person's skin for the best possible data of the person's heartbeat which also is the best utilization of this invention. Six heart monitoring wireless sensors [5] with their transmitting antennae [21] are fitted to this garment sending heartbeat signals to the electronic module [14] by the means of its antenna [21]. The defibrillation wired connectivity [9] shows two wet defibrillation pads [7] as located in two garment containment means [13] that provide the defibrillation shock means if the need arises. This wired connectivity [16] shows a separable flat wired connector [17] that allows unplugging of the undergarment [3] from the outer garment which would likely contain the weight and bulk of the external electronic module [14] and the external power source [18]. The outer garment [12] provides the style and fashion of the fitted garment [2] to cover and mask the working mechanisms of the fitted undergarment [3] so that any person using this invention can enjoy a more normal lifestyle. Together these garments will serve the client well over time and provide a level of safety and security that has been otherwise unavailable. These garments can provide a level of comfort as well in their use, and these garments are able to incorporate any manufacturer's or distributor's devices that measure heartbeats and offer a defibrillation means in these garments.

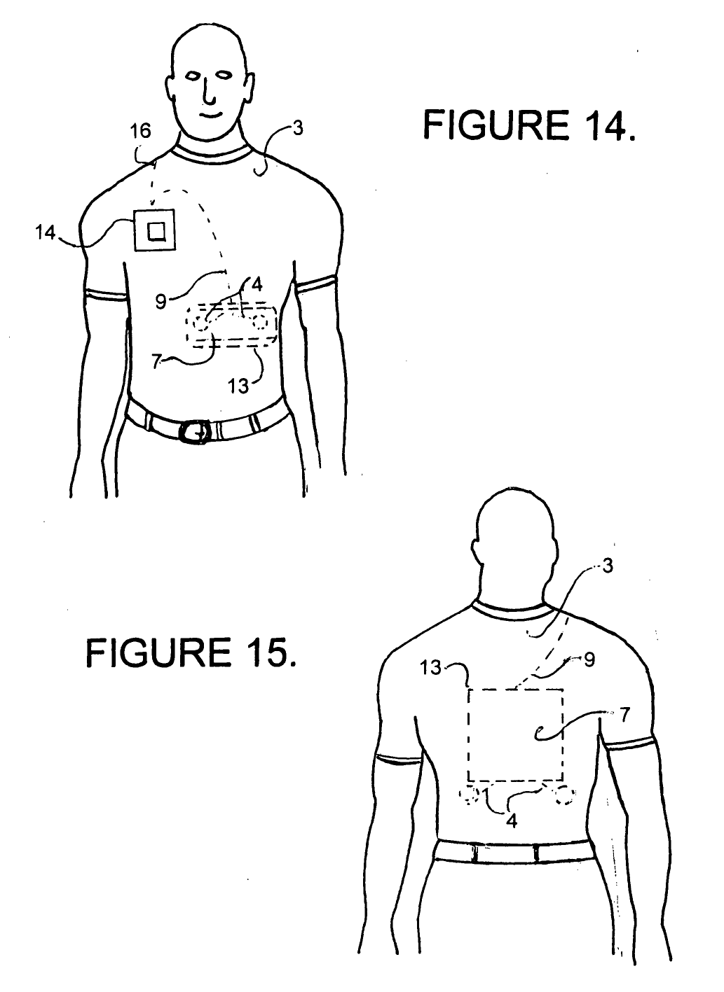

In the FIGS. 14 and 15 one sees a specialty device being incorporated in the fitted undergarment [3]. The fitted undergarment [3] offers a safe and comfortable means to support this specialty device with a cloth fitment that does not bind or chafe, and it can be used over long periods of time, and also, it can be washed when soiled. Additionally, the fitted undergarment [3] is able to be retrofitted to accept any and all of the electronic devices useful in heart monitoring and defibrillation. In this selected specialty device seen in FIGS. 14 and 15 the four heart sensors, two dry defibrillation pads that exude wet gel upon command, and their control module would be removed from the original belting and strapping framework, and then, to be refitted into this invention. In a manner to explain the ‘dry to wet’ defibrillation pads in that the dry means is the normal means, and the wet means is accomplished by a capacitive force upon the defibrillation signal to exude the wet gel through individual holes onto the person's skin to assure the best contact means for the defibrillation method. In FIG. 14 one sees the front wet defibrillation pad [7] secured in place by its garment containment means [13] wherein also two heart monitoring wired sensors [4] are shown incorporated in this front device. This device incorporates defibrillation wired connectivity [9] that connects to its electronic module [14] and the wired connectivity [16] extends to a rear device that is seen in the FIG. 15. In the fitted undergarment [3] the FIG. 15 shows the rear device operating two heart monitoring wired sensors [4] and the second wet defibrillation pad [7] with its wired connectivity [9], and the entire device secured in an garment containment means [13] and communication with its control module is provided through the wired means.

The best packaging for the fitter garment [2] would be two or more fitted undergarments [3], packaged with two or more of the stylish and fashionable outer garments [12] to offer the safety and security of full time heart monitoring with defibrillation capabilities. In packaging two or more of each type in a fitted garment [2] package there would be an opportunity for continued use as when soiled, each can be washed or dry cleaned, to restore full time use. In the fitted undergarment [3] strong and satisfactory heartbeat signals are optimal with its tight and direct contact to the person's skin, and in the integral outside wear outer garment [12] the garment containment means [13] would be an optimal placement to carry the weighty and somewhat bulky electronic module [14] and power source [18]. Thus, the best packaging would be several of each garment to give the best value and the best use for the fitted garment [2] to the user that desires the safest and most secure means for heart monitoring with defibrillation capabilities. A designed attachment of a defibrillator pad [6] inside of the fitted garment [3] can enable the creation of a pad that is covered by a membrane that is electrically conductive, but would require no wet gel means, and the wearer can enjoy a dry and comfortable cover surface to the fitted undergarment [3]

This invention can serve everyone with cardiac concerns, and serve every maker of cardiac monitoring equipment, and give selection and placement with continued support to every cardiac doctor and their cardiac facility.

This invention represents a new winning situation for the cardiac patient.

What is claimed is:

1. A fitted garment comprising the means of heart monitoring with defibrillation capabilities.

2. The fitted garment of claim 1 further comprising the means of heart monitoring comprising the means for multiple sensors to detect the heart beat.

3. The fitted garment of claim 2 further comprising said multiple sensors comprise said heart monitoring of said heart beat to a wearer borne module.

4. The fitted garment of claim 3 further comprising said multiple sensors comprise the means to deliver said heart monitoring of said heart beat by wireless means.

5. The fitted garment of claim 3 further comprising said wearer borne module would be fitted by containment means within said garment.

6. The fitted garment of claim 5 further comprising said wearer borne module comprises the means to transmit and receive its data externally.

7. The fitted garment of claim 5 further comprising said containment means comprises power means for said wearer borne module to transmit and receive its data externally.

8. The fitted garment of claim 7 further comprising the means to alert said wearer's cardiology facilities of the cardiac situation when said heart beat is irregular.

9. The fitted garment of claim 8 further comprising said wearer borne module comprise the means to continuously transmit said wearer's cardiac situation in such an emergency.

10. The fitted garment of claim 6 further comprising the means of said wearer borne module comprise the means to receive data to command said sensors to attempt to regulate said heart beat by a pacing means to return to a normal heat beat.

11. The fitted garment of claim 10 further comprising an adjustable power means comprising the means to initiate defibrillation means in the event of a sudden cardiac arrest to restart said wearer's heart.

12. A garment comprising the means to be fitted to a person comprising heart monitoring with defibrillation means.

13. The garment of claim 12 further comprising said garment comprises a fitted undergarment means for attachment of said heart monitoring means comprising defibrillation means.

14. The garment of claim 13 further comprising said heart monitoring means comprising defibrillation means comprises multiple sensors and pads attachable to said fitted undergarment.

15. The garment of claim 12 further comprising said fitted undergarment with containment means as requisite for medical devices and further comprising a separable outer garment with containment means.

16. The garment of claim 15 further comprising means for said outer garment with containment means and said undergarment with containment means to be integral in containment means for the requisite medical devices.

17. The garment of claim 16 further comprising means for said outer garment with containment means and said undergarment with containment means be separable and washable as requisite.

18. Garments comprising separable outer garment with containment means and separable undergarment with containment means comprises heart monitoring means with defibrillation means.

19. Garments of claim 18 further comprising separable garments with containment means for multiple heart sensors with defibrillation means comprise heart monitoring of said heart beat to a wearer borne module.

20. Garments of claim 19 further comprising said wearer borne module comprising the means to transmit and receive its data externally.
